(19) United States
(12) Patent Application Publication
(10) Pub. No.: US 2025/0017488 A1
(43) Pub. Date: Jan. 16, 2025

(54) SYSTEM AND METHODS FOR AUTOMATICALLY IDENTIFYING A BREATH VARIABILITY EVENT FROM PATIENT RESPIRATORY DATA ASSOCIATED WITH A MECHANICAL VENTILATOR

(71) Applicant: Breas Medical AB, Molnlycke (SE)

(72) Inventors: William A. Truschel, Oakmont, PA (US); Carl Van Loey, Edegem (BE); Ali Zorgani, Bron (FR)

(73) Assignee: Breas Medical AB, Molnlycke (SE)

(21) Appl. No.: 18/769,021

(22) Filed: Jul. 10, 2024

Related U.S. Application Data
(60) Provisional application No. 63/526,106, filed on Jul. 11, 2023.

(51) Int. Cl. A61B 5/08; A61M 16/00

(52) U.S. Cl. CPC A61B 5/0816; A61M 16/022; A61M 2016/0027; A61M 2016/0033

(57) ABSTRACT

A system and method are provided for identifying breath variability events from patient respiratory data obtained during the operation of a mechanical ventilator. The ventilator monitors and saves patient respiratory data during operation. The method includes extracting treatment parameters from a ventilation prescription, and using the treatment parameters to determine an Epoch size and a frequency band for analysis. An input signal (flow or pressure) is extracted from the patient data and from the input signal at least one parameter is extracted and analyzed. A Spectral Energy (Es) of the input signal is determined based on the signal parameter, an Epoch size and a frequency band and a Spectral Entropy (SE) is determined based on the Spectral Energy (Es). The Spectral Entropy may then be displayed in graphical form to identify breath variability events over the Epoch size. The method may further classify the type and degree of the events.

### CROSS-REFERENCE TO RELATED APPLICATIONS

This application claims the benefit of U.S. Provisional Patent Application Ser. No. 63/526,106 filed Jul. 11, 2023, the entire contents of which are incorporated herein by reference.

### BACKGROUND OF THE DISCLOSURE

### (1) Field of the Invention

The instant invention relates to mechanical ventilator systems, and more particularly to acquisition, collection and analysis of patient respiratory data associated with mechanical ventilation for identifying breathing irregularities.

### (2) Description of Related Art

Modern mechanical ventilators are sophisticated tools for both treating and monitoring patients with respiratory insufficiency and a variety of pathologies related to difficulty in breathing. The role of therapies in mechanical ventilation range from improving sleep to resolve daytime sleepiness to sustaining life in patients with little or no pulmonary muscular function.

The broad experience of clinicians using mechanical ventilation to treat patients has led to a great number of successes by improving the quality of life and delaying pathologic morbidity. An expert analysis of the many metrics standardly available on mechanical ventilators to be viewed, uploaded and downloaded showing a patient's breathing history has become the standard of care for both an assessment of the patient and the refinement of the mechanical ventilation prescription.

### SUMMARY OF THE DISCLOSURE

Until now, one particular set of metrics that may have far reaching diagnostic importance has been overlooked by the designers of mechanical ventilators, namely the degree of breathing variability.

The degree of breathing variability is a newer metric that is currently being studied as a potential predictor of success in mechanical ventilation. The present invention discloses methods for acquiring patient respiratory data, calculating breath variability metrics and displaying breath variability metrics as part of the diagnostic package in mechanical ventilation. The various methods and techniques developed are described herein along guidance for interpreting these variability metrics in patients who are undergoing mechanical ventilation therapy.

According to exemplary embodiments of the invention, a system and method are provided for identifying breath variability events from patient respiratory data obtained during the operation of a mechanical ventilator.

During operation of the ventilator system while configured according to a predetermined ventilation prescription, the mechanical ventilator continuously monitors and saves patient respiratory data from one or more integrated sensors. The method includes extracting predetermined treatment parameters from the prescription, and using the treatment parameters to determine an epoch and a frequency band for analysis.

An input signal (one of various flow or pressure signals) is extracted from the patient respiratory data and from the input signal at least one parameter is extracted and analyzed. The input signal may comprise a patient flow signal (Qp) in a pressure-controlled ventilation, a total flow signal (Qt) in a pressure-controlled ventilation, or a pressure signal (P) in a volume-controlled ventilation.

A Spectral Energy (Es) of the input signal is determined based on at least one signal parameter, the epoch and the frequency band. The Spectral Energy (Es) is resampled, and a Spectral Entropy (SE) is determined based on the resampled Spectral Energy (Es).

The Spectral Entropy is then displayed as a monitored variable in graphical form to visually depict one or more breath variability events over the epoch, based on the determined Spectral Entropy. The graphical form of the data may assist the clinician in visually identifying the magnitude and timing of respiratory patterns and irregularities in those respiratory patterns. Additionally, a Root Mean Square, standard deviation or variance of the input signal in the epoch may be determined and displayed as a relative measure of variability.

The method may include further algorithms for automatically identifying and classifying the variability events and setting alarm flags if determined to be irregularities.

While embodiments of the invention have been described as having the features recited, it is understood that various combinations of such features are also encompassed by particular embodiments of the invention and that the scope of the invention is limited by the claims and not the description.

### BRIEF DESCRIPTION OF THE DRAWINGS

While the specification concludes with claims particularly pointing out and distinctly claiming particular embodiments of the instant invention, various embodiments of the invention can be more readily understood and appreciated from the following descriptions of various embodiments of the invention when read in conjunction with the accompanying drawings in which:

FIG. 1 illustrates a ventilator system;

FIG. 2 illustrates lungs and the inspiration and expiration flows in and out of the lungs;

FIG. 3 illustrates a schematic of a programmable interventional ventilation system in accordance with the teaching of the present invention;

FIG. 4 illustrates the process of adding breath variability metrics to standard respiratory signal data sets;

FIG. 5 is a graphical illustration of events highlighted by the entropy signal (bottom) in a 9 hour data set (epoch); and

FIG. 6 illustrates an event classification from a simulated data set.

### DETAILED DESCRIPTION OF THE EXEMPLARY EMBODIMENTS

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like top, bottom, up, or down are used, they are not intended to limit the systems, devices, and methods disclosed herein. A person skilled in the art will recognize that these terms are merely relative to the system and device being discussed and are not universal.

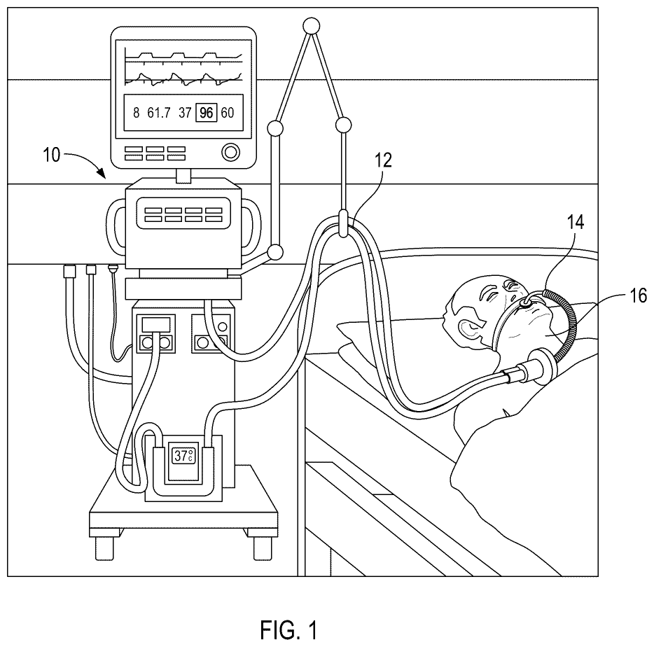

Turning now to the drawing figures, FIG. 1 illustrates a basic mechanical ventilator system 10 that provides pressurized air through the tube 12 into an airway adaptor 14, such as a tube or mask, to the user/patient 16. In some instances, a mask is not used, where the tube is directly fed into the trachea, such as a tracheostomy.

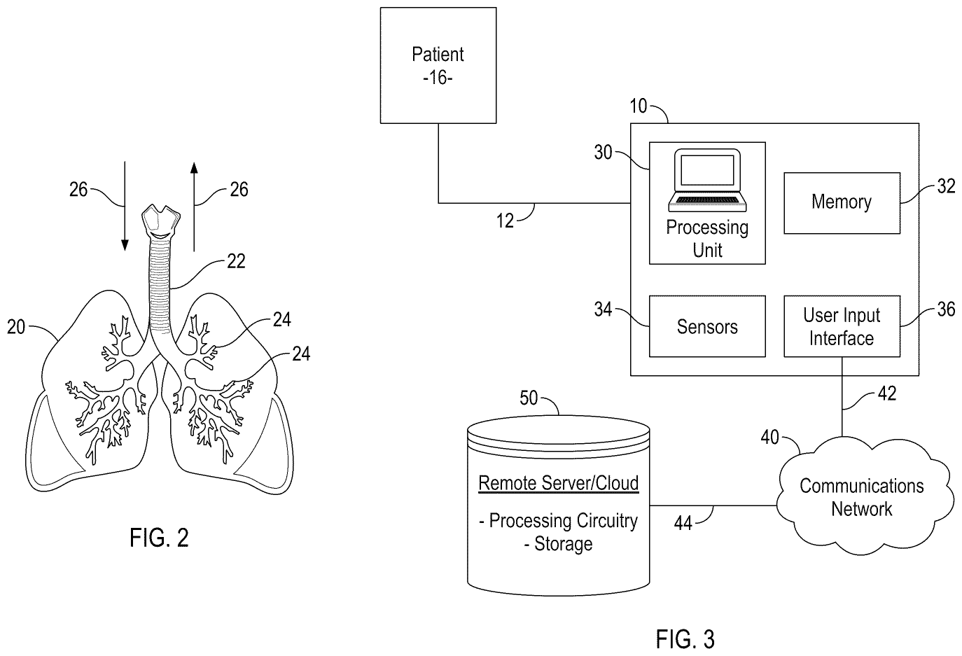

FIG. 2 illustrates lungs 20, including the trachea 22 and the bronchi of the lungs 24. The inspiration flow path 26 travels into the trachea 22 and into bronchi 24, whereas the expiration flow path 28 travels or flows out from the lungs 20 and bronchi 24 into and out of the trachea 22.

FIG. 3 illustrates a schematic of an interventional mechanical ventilation system 100 that includes a ventilator system 10, which includes a processing unit 30 configured to receive input operating parameters (as set forth hereinabove), via a user/patient input interface 36, implement intervention logic tuples and protocols, as well as direct and analyze sensor data captured by sensors 34, recall and place data into memory 32, and direct communications over a network 40 to remote server/cloud 50 that also includes processing circuitry and storage. Directed communications 42 can be made to and from the communications network, which can make directed communications 44 to and from the remote server/cloud 50. Cloud computing is generally understood in the art to mean the delivery of computing services, including servers, storage, databases, networking, software, analytics, and intelligence over the Internet (“the cloud”) to offer faster innovation, flexible resources, and economies of scale.

Variation in breathing is normal. The typical coefficient of variability in both breath size (Tidal Volume (Vt)) and breath rate (Respiratory Rate (RR)) tends to vary between 19 and 30% within a recorded epoch (time period). Factors that stimulate and produce variable breathing include both mechanoreceptors and chemoreceptors that respectively monitor stress and gas composition in the lung tissue, the movement of limbs, and voluntary brain activity.

A variability may be interpreted as natural and healthy, or in some cases it could mean a serious pathological complication. The interpretation of variability is complex and better understood with an analysis of the holistic scenario in which it is observed.

For example, breath variability is expected to change based on a human's sleep stage, e.g. REM versus non-REM. Exercise, age, and anxiety will affect the degree of variability. Within the context of this disclosure the focus is on the interpretation of the variability for patients who are undergoing Mechanical Ventilation (MV) treatment.

In acute patients with Acute Respiratory Distress Syndrome (ARDS) on mechanical ventilation, a statistically significant improvement in lung elastance, IL-6 production and various other markers improved when tidal volume (Vt) varied by 30%, but not when it varied by 15%, 7.5% or was invariable under controlled ventilation. This suggests that Vt variation should be included or suggested for monitoring or assessing patients with ARDS and/or lung injury.

Conversely, high variability in tidal volume exhibited in a patient undergoing respiratory assistance with mechanical ventilation may indicate patient-ventilatory asynchrony, such as flow starvation or paradoxical control. This suggests that Vt variation may be used to detect the degree of asynchrony and be used as a tool for adjusting ventilator settings.

In studies with Pediatric patients with anxiety disorders, greater variability positively correlated to arterial CO₂. Therefore, without the use of capnography, high variability may be an indication of hypercapnia in Non-Invasive Ventilation (NIV).

Patients with moderate to severe Chronic Obstructive Pulmonary Disease (COPD) have fewer sighs and therefore overall lower variability in breathing than healthy controls. A decrease in breathing variability may indicate a worsening of lung mechanics or dyspnea in COPD.

In Neuromuscular Disease (NMD) with Pressure Control, a decrease in daytime Vt variability may indicate lower inspiratory muscle strength and may be an early indicator of worsening vital capacity.

In patients with Restrictive Lung Disease (RLD), the variability of expiratory time was reduced by a factor of 27, the tidal volume variability was reduced by a factor of 12 and the variability of inspiratory time was reduced by a factor of 6 relative to healthy subjects. Subsequently, variability may be positively correlated to lung compliance in RLD. Restrictive lung disease (RLD) is a decrease in the total volume of air that the lungs are able to hold, which is often due to a decrease in the elasticity of the lungs.

In preterm infants with Chronic Lung Disease (CLD) resulting from immaturity, variability of volumetric CO2 was lower when compared to postmenstrual age infants. As a biomarker, the variability may be negatively correlated to alveolar recruitment in neonatal care of CLD with MV.

Decreased respiratory variability in critically ill adult patients has been shown to be predictive of poor outcome and even mortality.

The relationship between breath variability and disease is an untapped biomarker that in the modern world of data analytics may prove to be an important tool in the assessment of the MV patient condition or robustness of the MV prescription. However, breath variability is not currently a metric which is monitored.

The present invention provides a system for collecting relevant respiratory data and computing and presenting a novel method to indicate breath variability within the mechanical ventilation system.

A novel method for computing and presenting breath variability within MV is disclosed that both provides a metric of variability as well as an indicator when variability occurs. In addition to the novelty of the method, the novelty of providing breath variability metrics to a clinician on either the display of the MV, or in a data download, may offer additional insights for a clinician reviewing breath data for patient's undergoing MV treatment, and improve patient outcomes.

The invention will provide the following advantages over standard and typical breath data sets.

As described above, by quantifying a variability metric or degree of regularity in breath data clinicians or machines may determine patient health relative to disease state. (Note: In some diseases, regularity is healthy and in other diseases regularity with mechanical ventilation indicates greater morbidity.)

The method and presentation of breath variability on a time scale will highlight epochs with irregularities in breath data and may increase the efficiency of clinician waveform review.

Breath variability may be used to detect abnormal events in data sets and encourage further or closer inspection by a clinician to circumstances when breathing is variable. Breath variability may also highlight or be used to count events that can be resolved either through prescription or setting changes in MV.

A machine or clinician can learn through experience in viewing this time varying signal representing variability to classify breaths labeled as normal/hypopnea/tachypnea/asynchronous, etc. with machine learning techniques or careful observations to further enhance the insights provided by breath data.

### Summary of Quantitative Methods of Computing Breath Variability

There are several commonly used metrics to quantitatively represent a patient breath or breathing in MV.

Table 1 below illustrates a basic list of these metrics.

TABLE 1

| METRIC | ABBREVIATION | UNITS |
|---|---|---|
| Tidal Volume | Vt | Liters or ml |
| Respiratory Rate | RR | Breaths per Minute |
| Inspiratory Time | Tinsp | Seconds |
| Expiratory Time | Texp | Seconds |
| Ratio of Inspiratory | I:E Ratio | |
| to Expiratory Time | | |
| Minute Ventilation | Ve | Liters/min |
| Volumetric CO2 | VeCO2 or VCCO2 | ml or ml/min |
| End Tidal CO2 | ETCO2 | mm Hg or kPa |
| Pressure | P | cm H2O |
| Patient Flow | Qp | Liters per minute |
| Total Flow | Qc | Liters per minute |
| Leak | Ql | Liters per minute |

Some definitions and abbreviations that may be helpful for this application include the following:

Tidal Volume (Vt) is the amount of air the moves in or out of the lungs with each respiratory cycle.

Epoch is a distinctive period of time from which data can be gathered, recorded and analyzed. These can range from an hour, to hours, to a day, to days, to a week, to a month and so forth.

RR is the patient's respiratory breath rate usually expressed in breaths per minute indicating how many times per minutes the patient inhales and exhales.

Tinsp is the time during which the mechanical ventilator is delivering the inspiratory prescription. For synchronous ventilators, the inspiratory time matches the time during which a spontaneously breathing patient is contracting the diaphragmatic muscles to expand the lungs and draw air inwards.

Texp is the time during which the mechanical ventilator is delivering the expiratory prescription. For synchronous ventilators, the expiratory time matches the time during which the patient is expelling air outward or in a respiratory pause after all tidal air has been exhaled.

I:E ratio is the ratio of Tinsp to Texp, almost always expressed in the format 1:X where X is Texp/Tinsp.

Ve is the minute ventilation which represents the total accumulated exhaled air over the previous minute.

VeCO2 and VCCO2 is a value expressed in ml of the exhaled volume of only carbon dioxide gas in one breath.

ETCO2 is the highest partial pressure of CO2 measured in the exhaled gas.

Qp is the volumetric patient flow usually expressed in Liters per Minute and represents the time varying inward and outward rate of gas in the patient airways.

Qc is the total or circuit flow usually expressed in Liters per Minute and represents the time varying flow in the ventilator's breathing circuit. When leak is present in the patient interface or when the exhalation valve is not closed, the circuit flow is different from the patient flow, indicating that all flow in the circuit does not enter the patient airway.

Ql is the leak flow usually expressed in Liters per minute and represents the time varying difference in flow between the circuit flow and the patient flow. This value is typically averaged over one breath before it is displayed or recorded or a snapshot of the leak value is displayed at the expiratory pressure.

Patients on mechanical ventilation are usually monitored in an intensive care unit (ICU) and have monitors that measure several values related to respiration, including but not limited to: heart rate, respiratory rate (RR), blood pressure, Oxygen Saturation (SpO₂) End-Tidal Carbon Dioxide (ETCO₂), Minute Ventilation (Ve), Exhaled Tidal Volume (Vte), Static Lung Compliance (Cstat), Intrinsic PEEP (iPEEP), Apnea Hypopnea Index (AHI), Asynchrony Index (AI), Peak Inspiratory Flow (PIF), Peak Expiratory Flow (PEF), Percent of Spontaneous Triggers (% Spon), Static Lung Resistance (Rlung), Plateau Pressure (Pplat), Inspiratory to Expiratory Ratio (I:E Ratio), and Respiratory Rate Oxygenation (Rox).

Mechanical ventilators also measure parameters of operation including Pressure (P), Patient Flow (Qp), Total Flow (Qp) and Flow Leak (Ql). Accordingly, these metrics are readily available for use in computing breath variability.

Except for CO2, these tabular measurements can be continuously monitored by mechanical ventilators. The data from these measurements can be computed and stored for immediate alerts/alarms or later analysis, but within the context of this invention the machine combines a quantitative analysis of the variability of these metrics in the data.

The first choice in this methodology is to choose an epoch size appropriate for responsiveness to disease progression. This can be done over one size fits all or over a programmable epoch size ranging from 1 minute, 5 minutes, 1 hour or 1 day.

Once the epoch is decided, the variability can be computed in a number of ways.

Quantifying Variability within an Epoch

The simplest and most widely used measurement of variability is the coefficient of variation (CV).

The CV is the ratio the standard deviation of a data set relative to its mean. The CV has an advantage over a simple standard deviation and the coefficient of variance (Fano Factor) because it is relative to the data itself and independent of the units of measurement.

$$\\ \begin{matrix}CV = \frac{\sqrt{{\sum }_{N} {( {x}_{n} - \overline{x} )}^{2}}}{\overline{x}} & ( 1 )\end{matrix}$$

Where,
x̅ is the mean of the data in the epoch, and
N is the number of data points in the epoch.

In cardiology, variation is often measured as the Root Mean Squared of Successive Differences (RMSSD). This metric is an indication of the point by point variation in an ordinal set of data such as the number of milliseconds between successive heartbeats. This metric is rarely used for breath metrics, but researchers have correlated tidal volume variation in RMSSD to hypertension.

$$\\ \begin{matrix}RMSSD = \frac{\sqrt{{\sum }_{N} {( \vert {x}_{i - 1} - {x}_{i} \vert )}^{2}}}{N} & ( 2 )\end{matrix}$$

Proposed Novel Algorithm for the presentation of Breath Variability Theoretical Discerption:

The algorithm developed for this invention is based on the entropy computation of an input signal's spectral power. The input data can be: the Patient Flow (Qp) or Total Flow (Qc) signal in pressure-controlled ventilation, or the Pressure (P) signal in volume-controlled ventilation or other collected patient data.

The output signal called the Breath Entropy Metricity Signal will highlight the abnormalities present in the input signal over time for further analysis and interpretation.

### Signal Spectral Energy (Eₛ) Definition:

The energy Eₛ of a time variable signal x(t) is defined as the area under the curve of the squared magnitude of the input signal x(t);

$$\\ \begin{matrix}{E}_{s} = {\int }_{- ∞}^{+ ∞} {\vert x ( t ) \vert}^{2} dt & ( 3 )\end{matrix}$$

Also the spectral energy density of a signal x(t) is

$$\\ \begin{matrix}{E}_{sed} ( f ) = {\vert X ( f ) \vert}^{2} & ( 4 )\end{matrix}$$

Where X(f) is the Fourier Transform of the input signal x(t). To achieve this processing step we use a MATLAB function called (bandpower).

The “bandpower” function is a Matlab native function, designed to accurately compute the average power of an input signal, which is essential in various fields of signal processing. The primary purpose of this function is to provide a robust, efficient, and flexible tool for analyzing the power distribution within a signal over specified frequency ranges. This functionality is critical in applications where understanding the power characteristics of a signal is necessary for optimizing performance, diagnosing issues, or complying with regulatory standards. For biomedical signals such as electroencephalograms (EEG), electrocardiograms (ECG) or in this case a breath variability input signal, analyzing the power within specific frequency bands can help detect anomalies or patterns indicative of medical conditions. This may facilitate early diagnosis and monitoring of diseases.

By Parseval's theorem,

$$\\ \begin{matrix}{E}_{s} = \sum {E}_{sed} ( f ) & ( 5 )\end{matrix}$$

### Spectral Entropy (SE) Definition:

The spectral entropy (SE) of a signal is a measure of its spectral power distribution. The concept is based on the Shannon entropy, or information entropy, in information theory. The SE treats the signal's normalized power distribution in the frequency domain as a probability distribution and calculates the Shannon entropy of it. The Shannon entropy in this context is the spectral entropy of the signal. This property can be useful for feature extraction in fault detection and diagnosis. SE is also widely used as a feature in speech recognition and biomedical signal processing.

The equations for spectral entropy arise from the equations for the power spectrum and probability distribution for a signal. For a signal x(n), the power spectrum is S(m)=|X(m)|², where X(m) is the discrete Fourier transform of x(n). The probability distribution P(m) is then:

$$\\ \begin{matrix}P ( m ) = \frac{S ( m )}{{\sum }_{i} S ( i )} & ( 6 )\end{matrix}$$

The spectral Entropy H follows as:

$$\\ \begin{matrix}H = - {\sum }_{m = 1}^{N} P ( m ) {log}_{2} P ( m ) & ( 7 )\end{matrix}$$

To compute the instantaneous spectral entropy given a time-frequency power spectrogram S(t,f), the probability distribution at time t is:

$$\\ \begin{matrix}P ( t , m ) = \frac{S ( t , m )}{{\sum }_{f} S ( t , f )} & ( 8 )\end{matrix}$$

The spectral Entropy at time t is:

$$\\ \begin{matrix}H ( t ) = - {\sum }_{m = 1}^{N} P ( t , m ) {log}_{2} P ( t , m ) & ( 9 )\end{matrix}$$

To demonstrate this signal generation, we used a MATLAB function called (pentropy).

The “pentropy” function is a Matlab native function, designed to compute the spectral entropy of a signal or spectrum. Spectral entropy is a measure of the signal's complexity and can be used to analyze the distribution of power across different frequency components of the signal. This function is essential in various fields such as signal processing, audio analysis, biomedical engineering, and mechanical diagnostics. It provides a versatile tool for understanding the informational content and regularity of signals, which is crucial for applications that require signal characterization, anomaly detection, and feature extraction. In biomedical signal analysis, such as EEG or ECG and in this case breath variability, spectral entropy can detect abnormalities by highlighting changes in the complexity of the signal. This may also facilitate early diagnosis and monitoring of medical conditions.

### Algorithm Detailed Description:

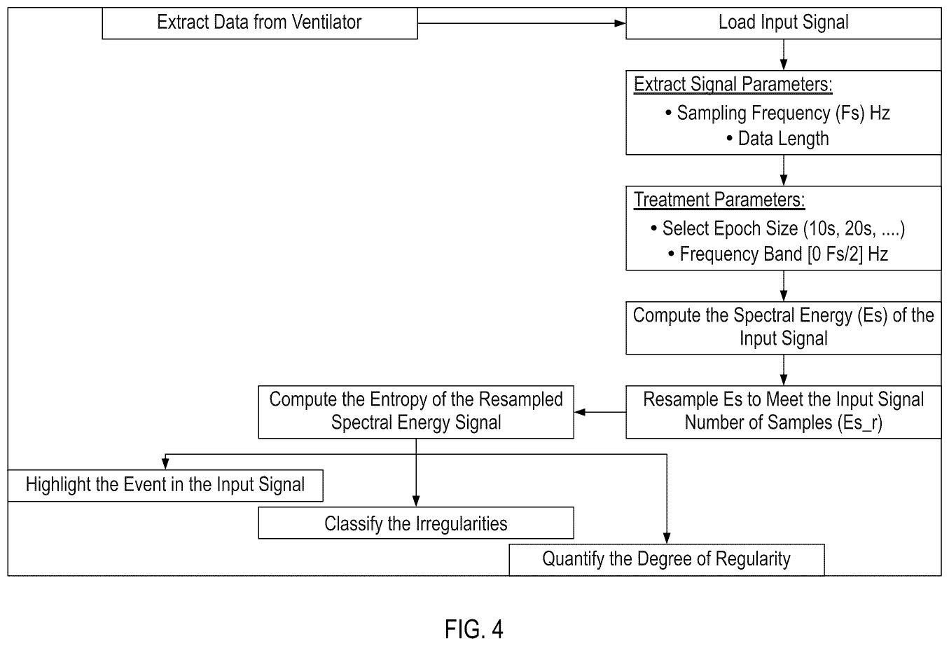

A block diagram is illustrated in FIG. 4 explaining the process of adding breath variability metrics to standard respiratory signal data sets.

### Highlighting the Breath Variability Events in a Large Data Set

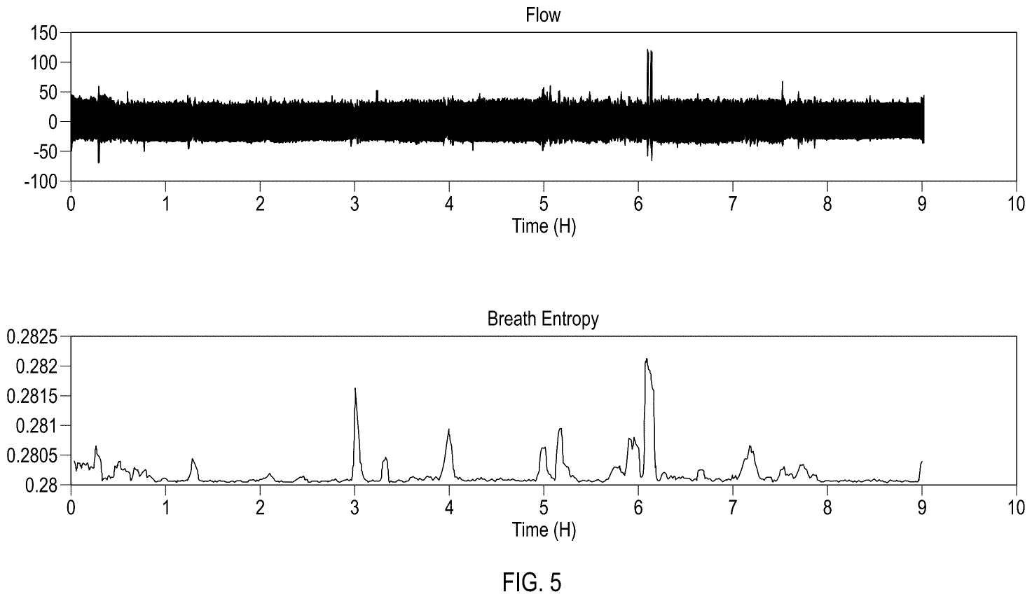

Turning to FIG. 5, there is illustrated a graphical depiction of a large data set showing a 9 hour epoch data set extracted from a real patient recording. In top portion of this illustration, it can be seen that the flow signal contains some distinct variabilities, some of which are clearly visible and others which are not so obvious. The entropy signal (SE) in the bottom portion (computed from the input flow signal above) highlights all the events present in the flow signal, the bottom signal highlights events that are present but not visible on the top signal.

### Classifying Irregularities in a Simulated Data Set

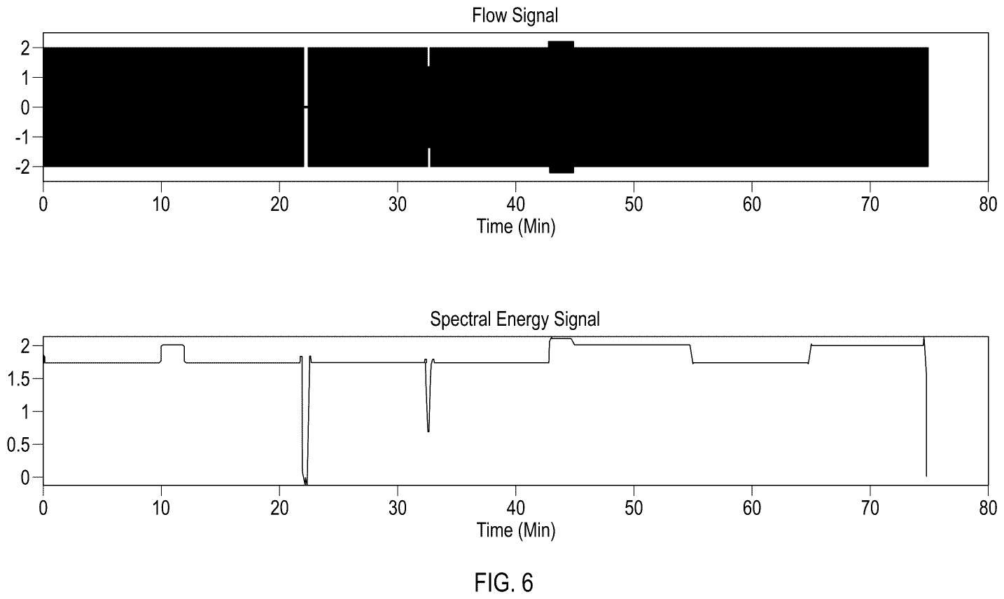

With the algorithms described herein it is possible to classify breath variability events based on energy level measurement. To do so, we use the spectral energy signal computed one step before the entropy signal (See FIG. 6).

Looking at the flow signal in FIG. 6, the nature of the event can be classified based on energy, wherein zero energy is an apnea, low energy is a hypopnea, and high energy is hyperventilation, Tachypnea, or high pressure (obstruction in volume modes) (see Table 2 below). The duration of each event can also be measured, and it can be compared to other events for relative severity of the event or for use as a filter to highlight events based on duration or amplitude, or to set alarms.

TABLE 2
Energy

| | High | Low | Zero |
|---|---|---|---|
| Patient Flow | Hyperventilation | Hypopnea | Apnea |
| Total Flow | High Leak | Blocked | Obstructed |
| | | Exhalation Port | Inhalation |
| | | | Limb |
| Pressure | Worsening | Missed Triggers, | Disconnection |
| | Respiratory | Flow Starvation, | |
| | Mechanics, Auto | High Leak | |
| | Triggering, | | |
| | Asynchrony | | |

### Quantifying a Degree of Irregularity

To quantify the degree of irregularities we use different statistical tools, such as Root Mean Square (RMS), Standard Deviation (STD) or median value. To illustrate that in this document we use the Standard Deviation (STD) value between a normal breathing phase (from 0 min to 3 h) on the flow signal from FIG. 6, and a more irregular breathing (between 3 h and 6 h). The standard deviation value of a normal breathing is 1.14 units and for the irregular breathing is 3.49 units. Thus, the STD can be used to quantify the degree of irregularities.

According to exemplary embodiments of the invention, a method for identifying breath variability events from patient respiratory data obtained during the operation of a mechanical ventilator comprises the steps of:
during operation of the ventilator system while configured according to a predetermined prescription, continuously monitoring and saving patient respiratory data from one or more sensors;
extracting predetermined treatment parameters from the prescription, and using the treatment parameters to determine an epoch and a frequency band;
extracting from the patient respiratory data an input signal;
extracting from the input signal, at least one signal parameter;
determining a Spectral Energy (Es) of the input signal based on the at least one signal parameter, epoch and a frequency band;
resampling the Spectral Energy;
determining a Spectral Entropy (SE) based on the resampled Spectral Energy (Es); and
displaying the Spectral Entropy in graphical form to visually identify one or more breath variability events over the epoch, based on the determined Spectral Entropy.

In some embodiments, the method may include:
a. determining the RMS, standard deviation or variance of the input signal in the epoch; and
b. displaying the RMS, standard deviation or variance as a relative measure of variability.

In some embodiments, the method may further comprise the step of analyzing the determined Spectral Entropy to automatically identify and classify said one or more breath variability events as an irregularity.

In some embodiments, the method may further comprise the step of analyzing the determined Spectral Entropy to quantify the degree of regularity associated with one of the one or more breath variability events.

In some embodiments, the input signal may be selected from the group consisting of: a patient flow signal (Qp) in a pressure-controlled ventilation, a total flow signal (Qc) in a pressure-controlled ventilation, and a pressure signal (P) in a volume-controlled ventilation.

In some embodiments, the system may include appropriate display apparatus wherein the method may further comprise the step of displaying the flow signals and associated computed spectral energy and providing a classification associated with the one or more identified events.

In some embodiments, the method may further comprise the step of isolating the input signal that is determinative of a breath variability event and visually displaying the isolated portion of the load input signal.

A ventilator system for providing mechanical ventilation to a target person according to a prescription may be provided as illustrated in FIG. 3 and configured to operate in accordance with the methods described herein.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A method for identifying breath variability events from patient respiratory data obtained during the operation of a mechanical ventilator; the method comprising the steps of:
during operation of the ventilator system while configured according to a predetermined prescription, continuously monitoring and saving patient respiratory data from one or more sensors;
extracting predetermined treatment parameters from the prescription, and using the treatment parameters to determine an epoch and a frequency band;
extracting from the patient respiratory data an input signal;
extracting from the input signal, at least one signal parameter;
determining a Spectral Energy (Es) of the input signal based on the at least one signal parameter, epoch and a frequency band;
resampling the Spectral Energy;
determining a Spectral Entropy (SE) based on the resampled Spectral Energy (Es); and
displaying the Spectral Entropy in graphical form to visually identify one or more breath variability events over the epoch, based on the determined Spectral Entropy.

2. The method of claim 1, further comprising the step of analyzing the determined Spectral Entropy to automatically identify and classify said one or more breath variability events as an irregularity.

3. The method of claim 1, further comprising the step of analyzing the determined Spectral Entropy to quantify the degree of regularity associated with one of the one or more breath variability events.

4. The method of claim 2, further comprising the step of analyzing the determined Spectral Entropy to quantify the degree of regularity associated with one of the one or more breath variability events.

5. The method of claim 1, wherein the input signal is selected from the group consisting of: a patient flow signal (Qp) in a pressure-controlled ventilation, a total flow signal (Qt) in a pressure-controlled ventilation, and a pressure signal (P) in a volume-controlled ventilation.

6. The method of claim 2, wherein the input signal is selected from the group consisting of: a patient flow signal (Qp) in a pressure-controlled ventilation, a total flow signal (Qt) in a pressure-controlled ventilation, and a pressure signal (P) in a volume-controlled ventilation.

7. The method of claim 3, wherein the input signal is selected from the group consisting of: a patient flow signal (Qp) in a pressure-controlled ventilation, a total flow signal (Qt) in a pressure-controlled ventilation, and a pressure signal (P) in a volume-controlled ventilation.

8. The method of claim 4, wherein the input signal is selected from the group consisting of: a patient flow signal (Qp) in a pressure-controlled ventilation, a total flow signal (Qt) in a pressure-controlled ventilation, and a pressure signal (P) in a volume-controlled ventilation.

9. The method of claim 2, further comprising the step of displaying the one or more identified events and providing a classification associated with the one or more identified events.

10. The method of claim 4, further comprising the step of displaying the one or more identified events and providing a classification associated with the one or more identified events.

11. The method of claim 6, further comprising the step of displaying the one or more identified events and providing a classification associated with the one or more identified events.

12. The method of claim 2, further comprising the step of isolating the input signal that is determinative of a breath variability event and visually displaying the isolated portion of the load input signal.

13. The method of claim 4, further comprising the step of isolating the input signal that is determinative of a breath variability event and visually displaying the isolated portion of the load input signal.

14. The method of claim 6, further comprising the step of isolating the input signal that is determinative of a breath variability event and visually displaying the isolated portion of the load input signal.

15. The method of claim 1 further comprising the steps of:
determining the RMS, standard deviation or variance of the input signal in the epoch; and
displaying the RMS, standard deviation or variance as a relative measure of variability.

16. The method of claim 2 further comprising the steps of:
determining the RMS, standard deviation or variance of the input signal in the epoch; and
displaying the RMS, standard deviation or variance as a relative measure of variability.

17. The method of claim 3 further comprising the steps of:
determining the RMS, standard deviation or variance of the input signal in the epoch; and
displaying the RMS, standard deviation or variance as a relative measure of variability.

18. A ventilator system for providing mechanical ventilation to a target person according to a prescription, configured to operate in accordance with the method of claim 1.

19. A ventilator system for providing mechanical ventilation to a target person according to a prescription, configured to operate in accordance with the method of claim 2.

20. A ventilator system for providing mechanical ventilation to a target person according to a prescription, configured to operate in accordance with the method of claim 3.
